(19) United States
(12) Patent Application Publication
(10) Pub. No.: US 2011/0125240 A1
(43) Pub. Date: May 26, 2011

(54) BIOCOMPATIBLE INDUCTOR FOR IMPLANTABLE LEAD AND METHOD OF MAKING SAME

(75) Inventors: Yong D. Zhao, Simi Valley, CA (US); Xiaoyi Min, Thousand Oaks, CA (US); Virote Indravudh, Santa Clarita, CA (US); Ingmar Viohl, Canyon Country, CA (US); J. Christopher Moulder, Portland, OR (US); Kevin L. Morgan, Simi Valley, CA (US); Phong D. Doan, Stevenson Ranch, CA (US)

(73) Assignee: PACESETTER, INC., Sylmar, CA (US)

(21) Appl. No.: 12/622,847

(22) Filed: Nov. 20, 2009

(51) Int. Cl. A61N 1/05

(52) U.S. Cl. 607/116

(57) ABSTRACT

A biocompatible inductor for an implantable medical lead is disclosed herein. In one embodiment the biocompatible inductor may include a biocompatible bobbin and a wire wound about a barrel of the biocompatible bobbin to form a coil. The wire may include an electrically conductive core, an electrically conductive biocompatible jacket extending over the core, and a coating of high dielectric strength insulation material extending over the jacket. Additionally, the biocompatible inductor may include medical adhesive located in gaps within the coil and a polyester shrink tube covering the coil.

### FIELD OF THE INVENTION

The present invention relates to medical apparatus and methods. More specifically, the present invention relates to a biocompatible inductors and methods of manufacturing the same.

### BACKGROUND OF THE INVENTION

Existing implantable medical leads for use with implantable pulse generators, such as neurostimulators, pacemakers, defibrillators or implantable cardioverter defibrillators (“ICD”), are prone to heating and induced current when placed in the strong magnetic (static, gradient and RF) fields of a magnetic resonance imaging (“MRI”) machine. The heating and induced current are the result of the lead acting like an antenna in the magnetic fields generated during a MRI. Heating and induced current in the lead may result in deterioration of stimulation thresholds or even increase the risk of cardiac tissue damage.

Over fifty percent of patients with an implantable pulse generator and implanted lead require, or can benefit from, an MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI are only going to grow over time. For example, MRI has been proposed as a visualization mechanism for lead implantation procedures.

There is a need in the art for an implantable medical lead configured for improved MRI safety. There is also a need in the art for methods of manufacturing and using such a lead. One method of producing such a lead is to block high frequency currents in the lead using an inductor.

### BRIEF SUMMARY OF THE INVENTION

A biocompatible inductor for an implantable medical lead is disclosed herein. In one embodiment the biocompatible inductor may include a biocompatible bobbin and a wire wound about a biocompatible bobbin to form a coil. The wire may include an electrically conductive core, an electrically conductive biocompatible jacket extending over the core, and a coating of high dielectric strength insulation material extending over the jacket. Additionally, the biocompatible inductor may include medical adhesive located in gaps within the coil and a polyester shrink tube covering the coil.

An implantable medical lead is disclosed herein. In one embodiment the implantable medical lead may include a body having a distal portion with an electrode and a proximal portion with a lead connector end. Additionally, the lead may include an electrical pathway extending between the electrode and lead connector end, the pathway including a first biocompatible coiled inductor. The first biocompatible inductor may include a biocompatible bobbin and a wire wound about a biocompatible bobbin to form a coil. The wire may include an electrically conductive core, a biocompatible electrically conductive jacket extending over the core, and a coating of high dielectric strength insulation material extending over the jacket. The coil may include medical adhesive located in gaps within the coil.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and Detailed Description are to be regarded as illustrative in nature and not restrictive.

### BRIEF DESCRIPTION OF THE DRAWINGS

FIG. 1A is an isometric view of a biocompatible inductor.

FIG. 1B is a transverse cross section of the wire.

FIG. 2A is an isometric view of a bobbin of the biocompatible inductor of FIG. 1.

FIGS. 2B-C illustrate a top view and a cross sectional view, respectively of the bobbin of FIG. 2A.

FIG. 3A is an isometric view of an inductor sub-assembly.

FIGS. 3B-C illustrate cross sectional views of alternative embodiments of the inductor sub assembly of FIG. 3A.

FIG. 4 is an isometric view of an implantable medical lead and pulse generator for connection thereto.

FIG. 5 is a longitudinal cross section of the lead distal end of the implantable medical lead of FIG. 4.

FIG. 6 is a plot of RF characterization measurement data of frequency versus impedance for a five-layer inductor.

### DETAILED DESCRIPTION

Disclosed herein is a biocompatible inductor 10 that may be used as an MRI RF heating filter in an implantable medical lead 100. In one embodiment, the biocompatible inductor may be a lumped inductor 10 that may be located near a distal end of the lead 100. The lumped inductor 10 may be made of multiple layers of biocompatible materials. In one embodiment, all the materials used for the biocompatible inductor 10 are biocompatible. Advantages provided by biocompatible inductor 10 may include reduced size relative to conventional lumped inductors used in the implantable medical leads, lower DC resistance, and self-resonant frequency close to 64 MHz or 128 MHz with impedance from 800 Ohms to over 20 kOhms.

Conventional inductors may be tightly wound coils of insulated copper or silver wires that have high electrical conductivity. To achieve the self resonant frequency (SRF) close to 64 MHz or 128 MHz and have sufficiently high impedance (usually greater than 10000), the coil is wound with many turns in multiple layers. The insulated wire and multilayered tight winding can generate strong mutual inductance and parasitic capacitance between the tight coil turns and coil layers. If this kind of inductor is installed onto the Brady, ICD, and CRT leads, the inductor is encapsulated well using a hermetic packaging. The inductor package is usually large in size and less reliable.

Large DC resistance generated by the long and small diameter wire is another concern as it may generate higher heating during the large current surge pulse tests that simulate external defibrillator shocks. The requirement of lower DC resistance also limits the replacement of the copper or silver wires using other biocompatible metals that have less electrical conductivity and will generate larger DC resistance.

The present disclosure includes designs of the lumped inductor of multiple layers using biocompatible materials. The use of biocompatible materials allows for a reduction in the size of the inductors such that they may be used in CRT and ICD leads, for example. Specifically, enclosure of a non-biocompatible inductor results in a much larger sized inductor, albeit more sturdy. Additionally, the presently disclosed biocompatible inductor designs overcome the aforementioned electrical issues.

Prototype inductors made of biocompatible materials and small inductor leads using biocompatible materials have been built. The bench electrical characterizations, large current pulse shock (8 A@2 ms), and MRI scan tests of the prototypes have proven the feasibility of the biocompatible inductors as the tip and ring RF filters that can be installed in the bipolar, active fixation, Brady lead. The inductors can be modified further for application to the ICD and CRT leads. With the CRT and ICD leads, the implementation of dual or single inductor(s) is not limited to large current pulse shock (8 A@2 ms), since the protection circuit in the ICD device is “Open” as the detection of either external or internal shocks.

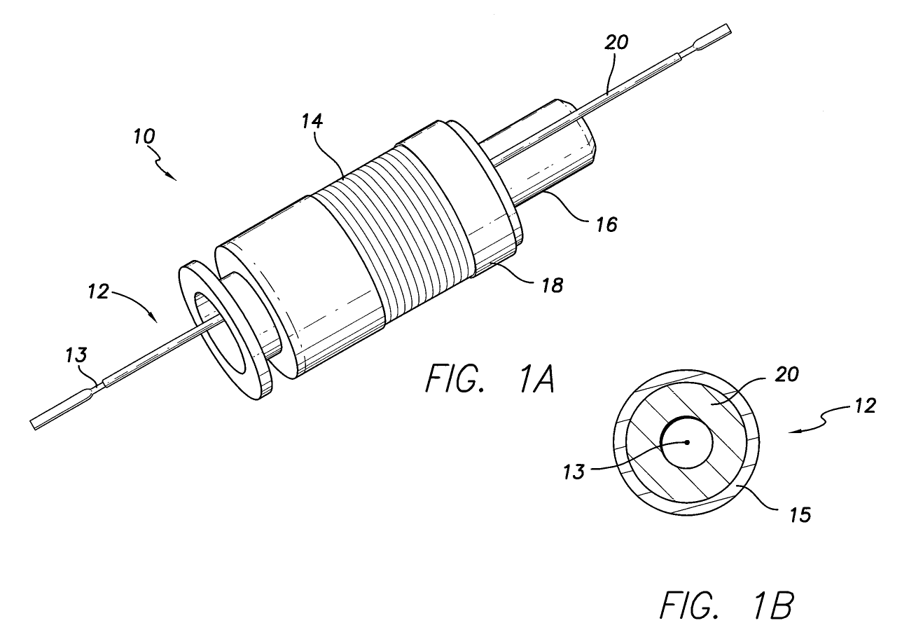

Turning to the figures and referring initially to FIG. 1A, an isometric view of the biocompatible inductor 10 is illustrated. Insulated wire 12 is tightly wound as single layer or multiple layer coils 14 over the bobbin 16 and may be either single filar or multiple filar. The bobbin 16 may be formed of an electrical insulation material such as polyetheretherkeytone (“PEEK”), or polyurethane, for example. As indicated in FIG. 1B, which is a transverse cross section of the wire 12, the wire 12 may be between approximately 0.001 of an inch to 0.005 of an inch in diameter with a core 13 of high electrical conductive material such as silver, gold, copper, with a jacket 20 of electrically conductive biocompatible material such as MP35N, Tantalum, Titanium, Platinum, Pt/Ir, etc. For example, 0.002 of an inch (or #44 gage) silver-cored MP35N may be used as the wire 12 and may be referred to as “DFT” wire. The DFT wire 12 may alternatively be coated or jacketed with a high dielectric strength insulation material 15 of ethylene tetrafluoroethylene (“ETFE”), polytetrafluoroethylene (“PTFE”), perfluoroalkoxy (“PFA”), Polyimide, Polyurethane, etc. with a thickness range between approximately 0.0002 of an inch to 0.003 of an inch. For example, 0.0005 of an inch thick ETFE may jacket the DFT wire 12. The core conductive material 13 may be between approximately 20 percent to 90 percent of the wire cross section. For example, a cross section of approximately 50 percent to 75 percent Ag may be used. Additionally, in some embodiments, round or non-round (e.g., rectangular) cross section wires can be applied.

A medical adhesive (“MedA”), such as NuSil MED-200, for example, may be used to fill gaps in the multiple layer coil 14 and in between layers during winding. The MedA tightly bonds the coil turn layers during the winding process. Additionally, the MedA enhances heat transfer when compared to air being in the gaps and interstial volumes of the coils 14.

Shrink tubing 18 may be placed over the coils 14 to tightly secure the winding, prevent potential mechanical damage and prevent fluid from getting too close to the inductor coil and possibly altering the electrical characteristics of the coils 14. For example, the shrink tubing 18 may prevent fluid from entering into the coil 14 that may change the self resonant frequency of the inductor 10. The shrink tubing 18 may be installed before or after the MedA has cured. In some embodiments, a 0.0005 of an inch to 0.003 of an inch thick shrink tubing of polyester may be used. For example, in one embodiment, approximately 0.0015 of an inch thick shrink tubing having a shrink temperature between approximately 50 degrees to 80 degrees Celsius and an elongation break point of approximately 115 percent may be used.

In an alternative embodiment, the wound coil 14 may be encapsulated with, or embedded into, a block of dielectric material of ceramics, ETFE, PTFE, PFA, Polyimide, PEEK, Tecothane, Polyurethane, GORE, etc., with the two wire termination portions exposed. Additionally, the wound coil 14 may be sealed hermetically or non-hermetically. In one embodiment, the coil 14 may be sealed in non-conductive enclosure such as ceramic with gold braising enclosure. In one example, a low temperature co-fire process may be used to make a ceramic capsule. In an alternative embodiment, an insert mold approach may be used to encapsulate the filter with the polymer of PEEK, etc. In yet another embodiment, a coating or thin film wrapping approach may be employed with the ETFE, Polyimide, etc.

As illustrated in FIG. 1, the wire 12 is shown as being exposed at the ends so that it may be electrically coupled with other electrically conductive members of the medical lead 100. Specifically, the inductor 10 is terminated and joined with the lead conductor using specific joining technologies such that the inductor fine wires 12 are free of mechanical loading and relative motions. For example, crimping, welding, swaging, bonding, and/or soldering may be used to join the inductor terminals with the lead conductor. Additionally, a very small amount of silver in the wire joint may be covered with a polymer cap (not shown) and the shrink tubing of polyester

The number of coil turns per layer and number of layers may be defined for given bobbin dimensions by modeling and experimental tests to achieve the desired self resonant frequency (SRF) within the range between approximately 0.7 to 1.3 times the MRI scanner frequency of 64 MHz, 128 MHz, etc., impedance at the MRI scanner frequency in the range of approximately 800 Ohms to approximately 30 kOhms, and total DC resistance less than approximately 20 Ohms.

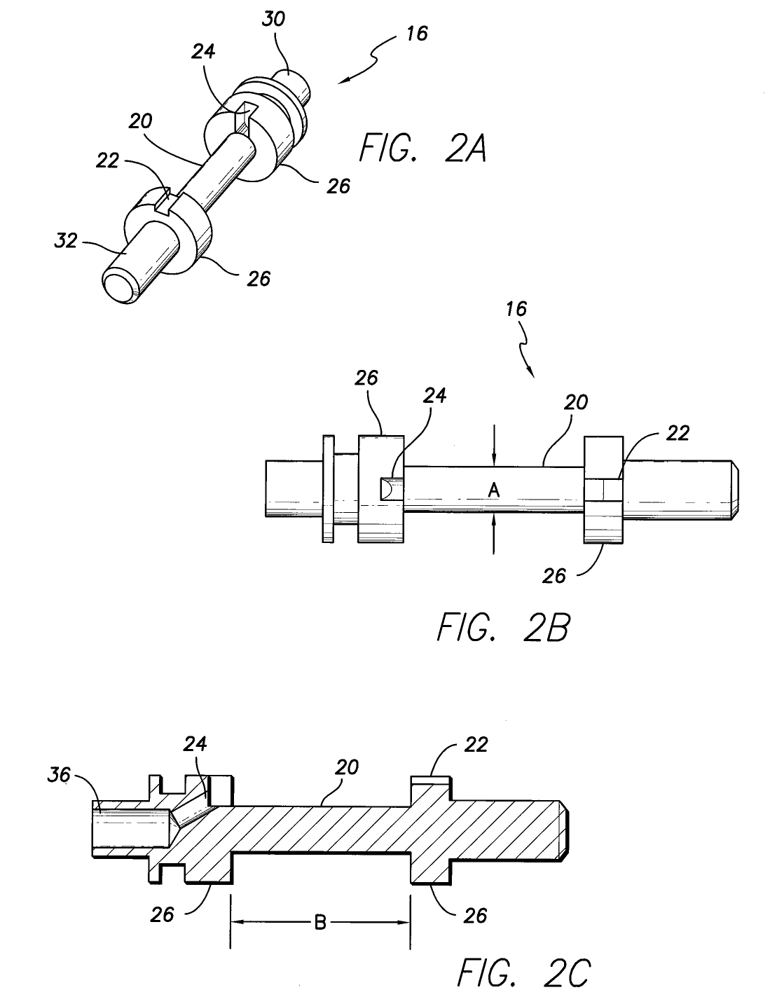

FIG. 2A is an isometric illustration of the bobbin 16 in accordance with an example embodiment of the present disclosure, and FIGS. 2B and 2C illustrate a top view and a cross sectional view, respectively, of the bobbin 16. The bobbin 16 may be a solid bar in one embodiment. In an alternative embodiment, the bobbin 16 may be a tube of round or non-round cross section. In any case, the bobbin 16 may be a tip inductor bobbin in the medical lead 100, as discussed in greater detail below.

As can be seen at arrow A, a barrel portion 20 of the bobbin 16 may be 0.015 of an inch through 0.060 of an inch in diameter and 0.050 of an inch through 0.300 of an inch in length. In one embodiment, the barrel portion may be approximately 0.022 of an inch in diameter and, at arrow B, approximately 0.093 of an inch in length. The DFT wire 12 is wound around the barrel portion 20 of the bobbin 16. Apertures 22 and 24 in flange structures 26 located at each end of the barrel region 20 allow for the wire 12 to be positioned within the barrel portion 22 and still interface other component parts of the lead 100, as will be discussed in greater detail below. Specifically, aperture 24 may allow for the wire 12 to pass through toward a proximal end 30 of the bobbin 16, while aperture 22 may allow for the other end of wire 12 to pass through toward the distal end 32 of the bobbin 16. The proximal end 30 of the bobbin 16 may be hollow and configured to receive other component parts of the medical lead 100.

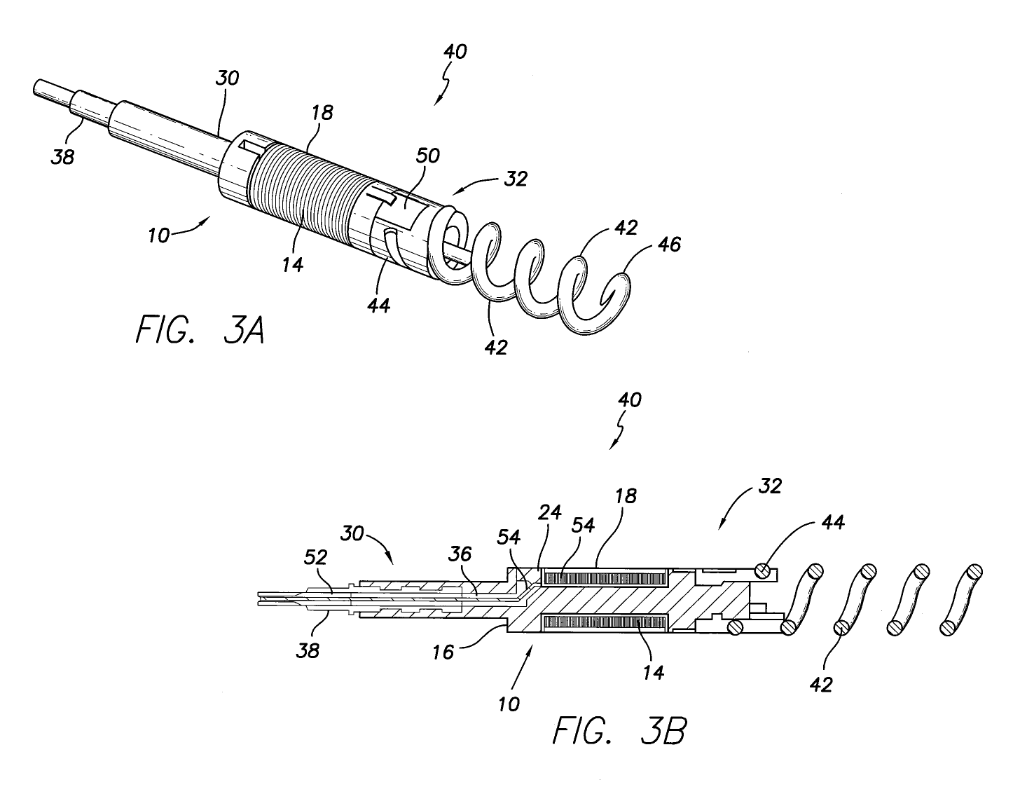

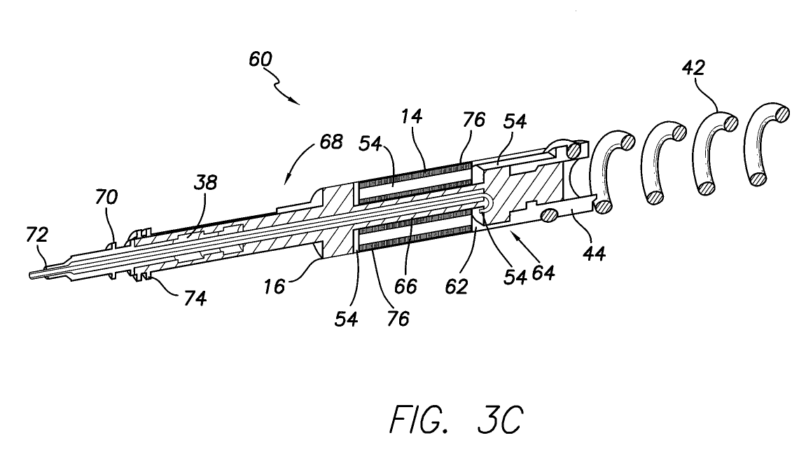

Specifically, for example, the proximal end 30 of the bobbin 16 may be configured to receive a MP35N shaft 38, as illustrated in FIGS. 3A and 3B. FIGS. 3A and 3B illustrate isometrical and cross sectional views, respectively, of an inductor sub-assembly 40 for use within the medical lead 100. The inductor sub assembly 40 includes the inductor 10 having the coil 14 of DFT wire 12. As discussed previously, the wire 12 may be either single or multiple filar and the coil 14 is wrapped in a polyester shrink wrap 18. Additionally, the inductor sub assembly 40 includes the MP35N shaft 38 coupled to the proximal end 30 of the inductor 10 and a helix assembly 42 coupled to the distal end 32 of the inductor 10.

The helix assembly 42 may include a base 44 and an anchor 46. The base 44 and the anchor 46 are mechanically and electrically coupled together. The distal portion 32 of the bobbin 16 may be received in the helix base 44 such that the bobbin 16 and the helix base 44 are mechanically coupled together. The base 44 may be formed of platinum, platinum-iridium alloy, MP35N, stainless steel, or etc. The helical anchor 46 may be formed of platinum, platinum-iridium alloy, MP35N, stainless steel, etc.

The terminal end of the wire 12 located at the distal portion 32 of the bobbin 16 may be welded to a platinum bracket 50. The bracket 50 is designed for the welding or crimping joining to meet both mechanical and electrical requirements. In one embodiment, the helix base may have a small hole that the wire can be inserted and the hole is then staked closed.

The terminal end of the wire 12 at the proximal portion 30 of the bobbin 16 may be fed though the aperture 24, through the hollow portion 36 of the proximal end 30 of the bobbin 16 to conductive epoxy 52 located within the shaft 38. MedA 54 may be potted in the aperture 24, as well as in the gaps and interstitial spaces of the coils 14. The epoxy and/or MedA potting increases the structural stability when subjected to severe loading during the manufacturing process, shipping and handling, as well as clinical applications. Additionally, the MedA potting completely seals the aperture so that there is no electrical leak from bobbin to coupler.

Whereas the embodiment of the inductor subassembly 40 illustrated in FIG. 3B allows for an odd number of coil layers to be wound about the bobbin 16, FIG. 3C illustrates a cross sectional view of an inductor subassembly 60 that allows for an even number of coil layers. To accomplish this, rather than having an aperture 24 located near the proximal end 30 of the bobbin 16, an aperture 62 is located near a distal portion 64 of the bobbin 16. The wire 12 may be fed through the aperture 62 and through a hollow portion 66 of the bobbin toward the proximal portion 68 of the bobbin 16, where the wire 12 may pass through a shaft 38. The proximal end 72 of the shaft 70 may be welded or crimped and have joining filler.

As shown in FIG. 3C, a sealing ring 74 may be installed at the junction between the shaft 70 and the bobbin 16. In other respects, the bobbin 16 and the shaft 70 are similar to the shaft 38 and bobbin 16 illustrated in FIG. 3B. Specifically, the coil 14 may be wound about the bobbin 16, shrink tube 76 may be placed over the coil 14, the helix assembly 42 may be coupled to a distal portion of the bobbin 16 and MedA 54 may be placed in the bobbin coil corners, gaps, and between coil layers. In both embodiments shown in FIGS. 3B and 3C, the bobbin cross section can be round or rectangular.

With the different embodiments, the coil layer number can be either even or odd and the inductor 10 may be used as a tip indictor in the medical lead 100. For the tip inductor, the inductor coil ID range may be approximately 0.015 to 0.050 of an inch, the coil length may be approximately 0.050 to 0.150 of an inch, and the total coil turns may be in the range of approximately 80 to 300, depending on the layer number and coil length.

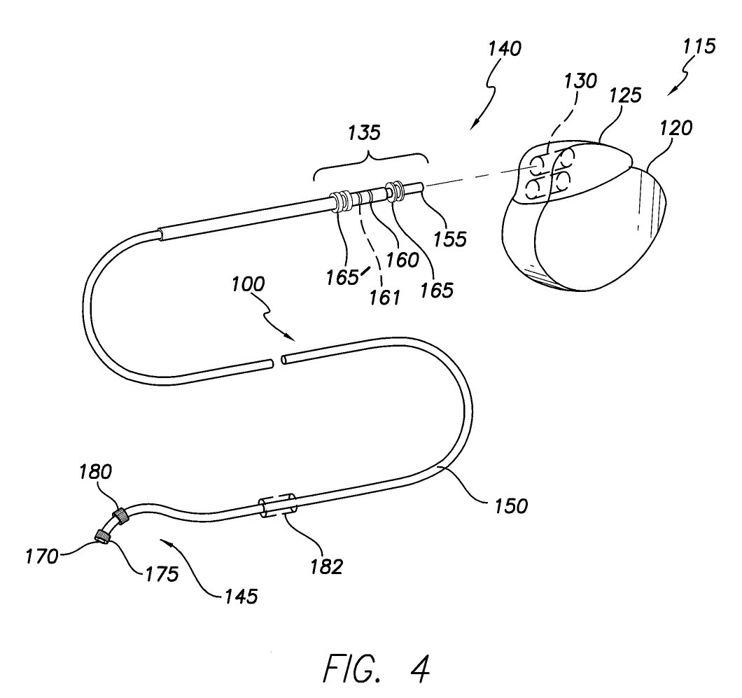

FIG. 4 is an isometric view of the lead 100 employing the biocompatible inductor 10 and a pulse generator 115 for connection thereto. The pulse generator 115 may be a pacemaker, defibrillator, ICD or neurostimulator. As indicated in FIG. 4, the pulse generator 115 may include a can 120, which may house the electrical components of the pulse generator 115, and a header 125. The header may be mounted on the can 120 and may be configured to receive a lead connector end 135 in a lead receiving receptacle 130.

As shown in FIG. 4, in one embodiment, the lead 100 may include a proximal end 140, a distal end 145 and a tubular body 150 extending between the proximal and distal ends 140 and 145. In some embodiments, the lead 100 may be a 6 French, model 1688T lead, as manufactured by St. Jude Medical of St. Paul, Minn. In other embodiments, the lead 100 may be a 6 French model 1346T lead, as manufactured by St. Jude Medical of St. Paul, Minn. In other embodiments, the lead 100 may be of other sizes and models. The lead 100 may be configured for a variety of uses. For example, the lead 100 may be a RA lead, RV lead, LV Brady lead, RV Tachy lead, intrapericardial lead, etc.

The lead connector end 135 located at the proximal end 140 may include a pin contact 155, a first ring contact 160, a second ring contact 161, which is optional, and sets of axially separated projecting seals 165. In some embodiments, the lead connector end 135 may include the same or different seals and may include a greater or lesser number of contacts. The lead connector end 135 may be received in a lead receiving receptacle 130 of the pulse generator 115 such that the seals 165 prevent the ingress of bodily fluids into the respective receptacle 130 and the contacts 155, 160, 161 electrically contact corresponding electrical terminals within the respective receptacle 130.

The lead distal end 145 may include a distal tip 170, a tip electrode 175 and a ring electrode 180. In some embodiments, the lead body 150 is configured to facilitate passive fixation and/or the lead distal end 145 includes features that facilitate passive fixation. In such embodiments, the tip electrode 175 may be in the form of a ring or domed cap and may form the distal tip 170 of the lead body 150. The biocompatible inductor 10 may be integrated into the lead distal end 145.

Additionally, in some embodiments, the distal end 145 may include a defibrillation coil 182 about the outer circumference of the lead body 150. The defibrillation coil 182 may be located proximal of the ring electrode 180. The ring electrode 180 may extend about the outer circumference of the lead body 150, proximal of the distal tip 170. In other embodiments, the distal end 145 may include a greater or lesser number of electrodes 175, 180 in different or similar configurations.

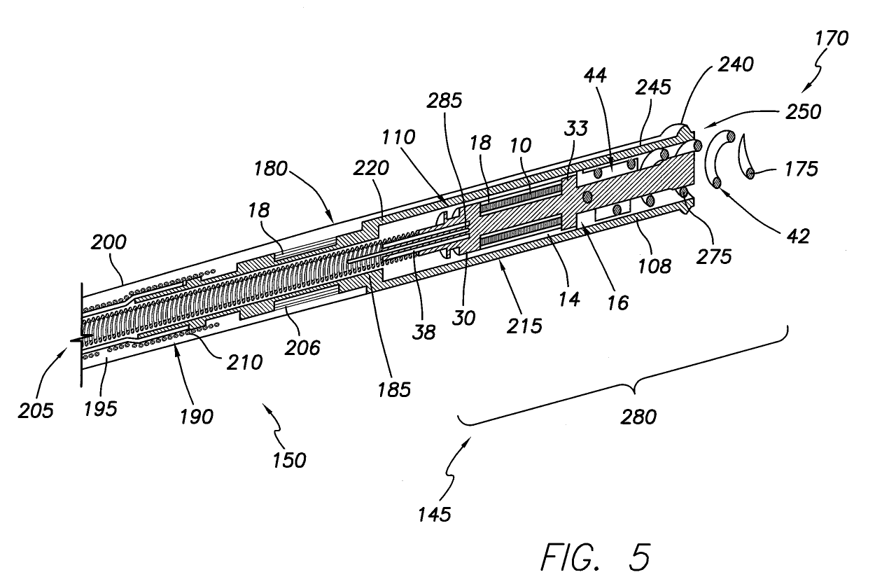

As illustrated in FIG. 5, which is a longitudinal cross-section of the lead distal end 145, in some embodiments, the tip electrode 175 may be in the form of the helical anchor 42 that is extendable from within the distal tip 170 for active fixation and serving as the tip electrode 46.

As can be understood from FIGS. 4 and 5, in one embodiment, the tip electrode 175 may be in electrical communication with the pin contact 155 via a first electrical conductor 185, and the ring electrode 180 may be in electrical communication with the first ring contact 160 via a second electrical conductor 190. In some embodiments, the defibrillation coil 182 may be in electrical communication with the second ring contact 161 via a third electrical conductor (not shown). In yet other embodiments, other lead components (e.g., additional ring electrodes, various types of sensors, etc.) (not shown) mounted on the lead body distal region 145 or other locations on the lead body 150 may be in electrical communication with a third ring contact (not shown) similar to the second ring contact 61 via a fourth electrical conductor (not shown). Depending on the embodiment, any one or more of the conductors 185, 190 may be a multi-strand or multi-filar cable or a single solid wire conductor run singly or grouped, for example in a pair.

As shown in FIG. 5, in one embodiment, the lead body 150 proximal of the ring electrode 180 may have a concentric layer configuration and may be formed at least in part by inner and outer helical coil conductors 185, 190, an inner tubing 195, and an outer tubing 200. The helical coil conductor 185, 190, the inner tubing 195 and the outer tubing 200 form concentric layers of the lead body 150. The inner helical coil conductor 185 forms the inner most layer of the lead body 150 and defines a central lumen 205 for receiving a stylet or guidewire therethrough.

Additionally, a ring inductor bobbin 206 may have a round or non-round cross section and may encircle a portion of the inner helical coil conductor 185 as well as the core lumen 205 to allow the tip conductor coil to pass through. Additionally, the ring inductor 206 may be located under the ring electrode 180. The ring inductor 206 may have an inductor coil ID range of approximately 0.035 of an inch to 0.070 of and inch. The coil length may further be approximately 0.030 of an inch to 0.120 of an inch with the total number of coil turns in the range of approximately 40 to 200, depending on the layer number and coil length.

The inner helical coil conductor 185 is surrounded by the inner tubing 195, which forms the second most inner layer of the lead body 150. The outer helical coil conductor 190 surrounds the inner tubing 195 and forms the third most inner layer of the lead body 150. The outer tubing 200 surrounds the outer helical coil conductor 190 and forms the outer most layer of the lead body 150. In one embodiment, the inner tubing 195 may be formed of an electrical insulation material such as, for example, ETFE), PTFE, silicone rubber, silicone rubber polyurethane copolymer (“SPC”). The inner tubing 195 may serve to electrically isolate the inner conductor 185 from the outer conductor 190. The outer tubing 200 may be formed of a biocompatible electrical insulation material such as, for example, silicone rubber, SPC, polyurethane, or GORE. The outer tubing 200 may serve as the jacket 200 of the lead body 150, defining the outer circumferential surface 210 of the lead body 150.

In one embodiment, the lead body 150 in the vicinity of the ring electrode 180 transitions from the above-described concentric layer configuration to a header assembly 215. For example, in one embodiment, the outer tubing 200 terminates at a proximal end of the ring inductor bobbin 206, the outer conductor 190 mechanically and electrically couples to a proximal conductive end of the ring inductor 206 that has a distal conductive end coupled to the ring electrode 180, the inner tubing 195 is sandwiched between the interior of the outer conductor 190 and the proximal end of the ring inductor 206, and the inner conductor 185 extends distally past the ring electrode 180 to electrically and mechanically couple to components of the header assembly 215 as discussed below.

As depicted in FIG. 5, in one embodiment, the header assembly 215 may include the body 220 and the inductor sub assembly 40, for example, including the coupler 38 and the helix assembly 42. The header body 220 may be a tube forming the outer circumferential surface of the header assembly 215 and enclosing the components of the assembly 215. The header body 220 may have a soft atraumatic distal tip 240 with a radiopaque marker 245 to facilitate the soft atraumatic distal tip 240 being visualized during fluoroscopy. The distal tip 240 may form the extreme distal end 170 of the lead 10 and includes a distal opening 250 through which the helical tip anchor 175 may be extended or retracted. The header body 220 may be formed of materials such as, PEEK, or polyurethane, for example, the soft distal tip 240 may be formed of silicone rubber or SPC, or other suitable material, and the radiopaque marker 245 may be formed of platinum, platinum-iridium alloy, tungsten, tantalum, or other suitable material.

Additionally, a helix nut 108 may also be provided near the distal end of the medical lead 100. The helix nut 108 causes the helix to extend or contract when the helix is rotated against it and the helix nut 108 also prevents the helix from over extending and extraction. A blood seal 110 may be provided near the proximal end of the bobbin 16 to prevent body fluids from accessing portions of the lead 100 beyond the bobbin 16. The blood seal of a soft polymer, such as Silicone, is placed between the two terminals of the inductor assembly, so as to prevent blood from forming a potential electrical bypass of the inductor circuit.

As illustrated in FIG. 5, the shrink tube 18 may extend about the inductor 10 to generally enclose the inductor 10 within the boundaries of the bobbin 16 and the shrink tube 18. The shrink tube 18 may act as a barrier between the inductor 10 and the inner circumferential surface of the header body 220. Also, the shrink tube 18 may be used to form at least part of a hermitic seal about the coil inductor 10. The shrink tube 18 may be formed of fluorinated ethylene propylene (“FEP”), polyester, or etc.

As described above and as indicated in FIG. 5, the helix assembly 42 may include a base 44 and the helical anchor electrode 175. The base 44 forms the proximal portion of the assembly 42. The helical anchor electrode 175 forms the distal portion of the assembly 42. A steroid plug 275 may be located within the volume defined by the helical coils of the helical anchor electrode 175. The base 44 and the helical anchor electrode 175 are mechanically and electrically coupled together. The distal portion of the bobbin 16 may be received in the helix base 44 such that the bobbin 16 and the helix base 44 are mechanically coupled to each other. The base 44 of the helix assembly 42 may be formed of platinum, platinum-iridium alloy, MP35N, stainless steel, or etc. The helical anchor electrode 175 may be formed of platinum, platinum-iridium alloy, MP35N, or stainless steel, for example.

As illustrated in FIG. 5, a distal portion of the coupler 38 may be received in the proximal portion of the bobbin 16 such that the coupler 38 and bobbin 16 are mechanically coupled to each other. A proximal portion of the coupler 38 may be received in the lumen 205 of the inner coil conductor 185 at the extreme distal end of the inner coil conductor 185 such that the inner coil conductor 185 and the coupler 38 are both mechanically and electrically coupled to each other. The coupler 38 may be formed of MP35N, platinum, platinum-iridium, alloy, or stainless steel, for example. As can be understood from FIG. 5 and the preceding discussion, the coupler 38, inductor 10, and helix assembly 42 are mechanically coupled together such that these elements 38, 10, 42 of the header assembly 215 do not displace relative to each other. Instead these elements 38, 10, 42 of the header assembly 215 are capable of displacing as a unit relative to, and within, the body 220 when a stylet or similar tool is inserted through the lumen 205 to engage the coupler 38. In other words, these elements 38, 10, 42 of the header assembly 215 form an electrode-inductor assembly 280, which can be caused to displace relative to, and within, the header assembly body 220 when a stylet engages the proximal end of the coupler 38. The helix nut causes the helix to extend. In one embodiment, the stylet may be inserted into the lumen 205 and used to stabilize and locate the lead. Turning the connector pin causes the inner coil and helix to rotate and the rotation of helix against the helix nut causes the helix to extend.

As already mentioned and indicated in FIG. 5, the coils 14 may be wound about the barrel portion of the bobbin 16. A proximal end 285 of the coils 14 may extend through the proximal portion 30 of the bobbin 16 to electrically couple with the coupler 38, and a distal end 32 of the coils 14 may extend through the distal portion of the bobbin 16 to electrically couple to the helix base 44. Thus, in one embodiment, the coil inductor 10 is in electrical communication with the both the inner coil conductor 185, via the coupler 38, and the helical anchor electrode 175, via the helix base 44. Therefore, the coil inductor 10 acts as an electrical pathway between the coupler 38 and the helix base 44. In one embodiment, all electricity destined for the helical anchor electrode 46 from the inner coil conductor 185 passes through the coil inductor 10 such that the inner coil conductor 185 and the electrode 176 both benefit from the presence of the coil inductor 10, the coil inductor 10 acting as a high impedance in a magnetic field of an MRI.

A similar situation may exist with respect to the ring inductor 206 and the outer conductor 190. For example, the coils may be wound about the barrel portion of the bobbin of the ring inductor 206. A proximal end of the coils may extend through the proximal portion of the bobbin of the ring inductor 206 to electrically couple with the outer conductor 190, and a distal end of the coils may extend through the distal portion of the bobbin of the ring inductor 206 to electrically couple to the ring electrode 180. Thus, in one embodiment, the coil inductor 206 is in electrical communication with the both the outer coil conductor 190 and the ring electrode 180. Therefore, the coil inductor 206 acts as an electrical pathway between the outer conductor 190 and the ring electrode 180. In one embodiment, all electricity destined for the ring electrode 180 from the outer coil conductor 190 passes through the coil inductor 206 such that the outer coil conductor 190 and the electrode 180 both benefit from the presence of the coil inductor 206, the coil inductor 206 acting as a high impedance in a magnetic field of an MRI.

As the helix base 44 may be formed of a mass of metal, the helix base 44 may serve as a relatively large heat sink for the inductor coil 14, which is physically connected to the helix base 44. Similarly, as the coupler 38 may be formed of a mass of metal, the coupler 38 may serve as a relatively large heat sink for the inductor coil 14, which is physically connected to the coupler 38.

In accordance with the foregoing description, a tip inductor of single filar, 5-layers, total 140-turns, coil ID of 0.022″ and coil length of 0.098″ was developed. The inductor body is rigid, because of the tightly bonded bobbin-coil, MedA, and shrink tubing, and thus the fine DFT wire is protected from mechanical damage when the inductor lead is implanted and in clinical service. A corresponding ring inductor 206 of single filar, 3-layers, total 65-turns, coil ID of 0.045″ and coil length of 0.077 of an inch was also developed. Both the tip and ring inductors are biocompatible and bio-stable. The tip inductor 10 is installed on the helix shaft inside the header of nonconductive polymer of PEEK, etc., and the ring inductor 206 is installed partially or completely inside the ring electrode. Alternatively, the ring inductor 206 may be installed partially or completely outside the ring electrode.

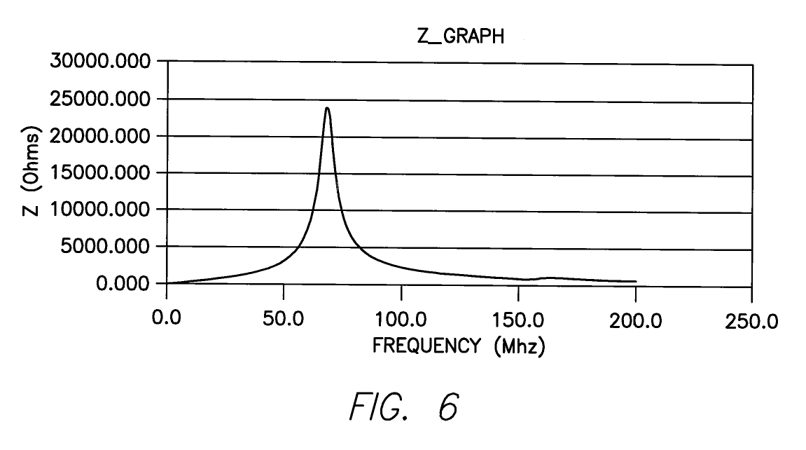

The tip and ring inductors can be installed in the St. Jude Medical lead model 1688T of 6 French with the SRF within 5% of 64 MHz, impedance in the range of 4.5 kΩ˜25.0 kΩ at the 64 MHz, and inductor DC resistance is less than 7Ω. The MRI RF heating is less than 3 degrees C. at both the tip and the ring. FIG. 6 illustrates the RF characterization measurement data for the 5 layer inductor (i.e. tip inductor) with impedance (Z) being a function of the frequency (MHz). As can be seen, at or near 64 MHz impedance is near 25kOhms.

Flexible and ductile DFT wires (the yield stress of approximately 39,109 psi and 75,121 psi, break load of approximately 0.201 lb and 0.236 lb, and elongation of approximately 11.7% and 15.4%, for the #44 gage (or 0.002″) 75% and 50% Ag cored MP35N wires, respectively) were used with the filled MedA (Durometer Type A is approximately 25 and break elongation is approximately 700% for the cured MED-2000) to absorb thermal expansion and contraction and, thus, enhance the structural reliability of the inductors under thermal shock. Additionally, the high percentage silver contented DFT wires and the PEEK bobbin can withstand the large current pulse shock, such as the 8 A for 2 ms to simulate the external defibrillator shock, primarily due to the lower DC resistance of the wire metals and the high service temperature of the insulation coating material (ETFE's melting point is approximately 267° C. or 512° F.), shrink tubing material (polyester's melting point is approximately 255 degrees Celsius or 490 degrees Fahrenheit), and bobbin material (PEEK's melting point is approximately 340° C. or 644° F.). Further, the polyester shrink tubing, MedA, and ETFE or polyimide coating or film have the properties and capabilities of low rate water absorption, which can ensure the performance stability of the inductor surrounded by body fluid.

What is claimed is:

1. A biocompatible inductor for an implantable medical lead comprising:
a biocompatible bobbin;
a wire wound about a barrel of the biocompatible bobbin to form a coil, wherein the wire comprises an electrically conductive core, a biocompatible electrically conductive jacket extending over the core, and a coating of high dielectric strength insulation material extending over the jacket;
medical adhesive located in gaps within the coil; and
a polyester shrink tube covering the coil.

2. The biocompatible inductor of claim 1, wherein the biocompatible bobbin comprises a solid bar or a hollow tube and comprises at least one of PEEK, Tecothane, Polyurethane, and GORE.

3. The biocompatible inductor of claim 1, wherein the wire has a diameter of 0.001 to 0.005 of an inch.

4. The biocompatible inductor of claim 1, wherein the core includes at least one of silver, gold and copper.

5. The biocompatible inductor of claim 3, wherein the core comprises between 20 and 90 percent of a cross section of the wire.

6. The biocompatible inductor of claim 3, wherein the biocompatible electrically conductive jacket comprises at least one of MP35N, Tantalum, Platinum, Titanium or Platinum-Iridium.

7. The biocompatible inductor of claim 6, wherein the biocompatible electrically conductive jacket comprises a thickness between 0.0002 and 0.003 of an inch.

8. The biocompatible inductor of claim 1, wherein the dielectric coating comprises at least one of ETFE, PTFE, PFA, Polyimide, and Polyurethane.

9. The biocompatible inductor of claim 1, wherein the wire has a round or rectangular cross section.

10. The biocompatible inductor of claim 1, wherein the wire is multiple filar.

11. The biocompatible inductor of claim 1, wherein the inductor has a self-resonant frequency within the range of 0.7 to 1.3 times an MRI scanner frequency of 64 MHz or 128 MHz.

12. The biocompatible inductor of claim 1, wherein a proximal portion of the biocompatible bobbin comprises a blood seal.

13. An implantable medical lead comprising:
a body including a distal portion with an electrode and a proximal portion with a lead connector end; and
an electrical pathway extending between the electrode and lead connector end, the pathway including a first biocompatible coiled inductor comprising:
a biocompatible bobbin;
a wire wound about a barrel of the biocompatible bobbin to form a coil, wherein the wire comprises:
an electrically conductive core;
a biocompatible electrically conductive jacket extending over the core; and
a coating of high dielectric strength insulation material extending over the jacket; and
medical adhesive located in gaps within the coil.

14. The implantable medical lead of claim 13, wherein the first biocompatible coiled inductor further comprises a polyester shrink tube or silicon tube covering the coil.

15. The implantable medical lead of claim 13, further comprising a second biocompatible inductor, the second biocompatible inductor defining a lumen through which a conductive member electrically coupled to the first biocompatible inductor may pass.

16. The implantable medical lead of claim 13, wherein the biocompatible bobbin comprises a solid bar or a hollow tube and comprises at least one of PEEK, Tecothane, Polyurethane, and GORE.

17. The implantable medical lead of claim 13, wherein the wire comprises a fine wire of between 0.001 and 0.005 of an inch diameter.

18. The implantable medical lead of claim 17, wherein the core comprises 20 to 90 percent of a cross section of the wire.

19. The implantable medical lead of claim 17, wherein the biocompatible jacket comprises at least one of MP35N, Tantalum, Platinum, Titanium or Platinum-Iridium.

20. The implantable medical lead of claim 19, wherein the biocompatible jacket comprises a thickness between 0.0002 and 0.003 of an inch.

21. The implantable medical lead of claim 13, wherein the core comprises at least one of silver, gold and copper.

22. The implantable medical lead of claim 13, wherein the dielectric coating comprises at least one of ETFE, PTFE, PFA, Polyimide, and Polyurethane.

23. The implantable medical lead of claim 13, further comprising a blood seal disposed at a proximal portion of the biocompatible bobbin.
